(19) United States
(12) Patent Application Publication
(10) Pub. No.: US 2020/0268346 A1
(43) Pub. Date: Aug. 27, 2020

(54) AN ULTRASONIC PROBE, AN ULTRASONIC IMAGING SYSTEM AND USE METHOD FOR BIOPSY NEEDLE VISUALIZATION ENHANCEMENT

(71) Applicant: IMSONIC MEDICAL CHINA, INC., Jiangsu (CN)

(72) Inventor: Xiaohui HAO, Jiangsu (CN)

(21) Appl. No.: 16/644,133

(22) PCT Filed: Sep. 26, 2018
(86) PCT No.: PCT/CN2018/107455

§ 371 Date: Mar. 3, 2020

(30) Foreign Application Priority Data
Aug. 7, 2018 (CN) 201810890035.5

(51) Int. Cl. A61B 8/00; A61B 8/12; A61B 8/08

(52) U.S. Cl. CPC A61B 8/4494; A61B 8/0841; A61B 8/4444; A61B 8/12

(57) ABSTRACT

This invention discloses an ultrasonic probe, an ultrasonic imaging system and their detail usage for biopsy needle visualization enhancement. The invention adds new lateral element arrays in the elevation direction of a center element array of a conventional linear/curve linear array probe, and allows a clinician to control the lateral element arrays to be turned on or off manually, or by a system analysis unit through intelligent judgment. During imaging, when the lateral element arrays are turned on, they will substantially enlarge the effective range of an ultrasonic field created by this probe in a needle body searching mode, so that the needle body is more easily captured and displayed in an image. By turning off the lateral arrays after the needle body is captured, a high-resolution imaging mode is recovered, thus the image quality will still be good enough for conventional clinical usage.

### BACKGROUND

### Technical Field

The present invention relates to the technical field of ultrasonic diagnosis and detection, and more particularly relates to an ultrasonic probe, as well as an ultrasonic imaging system and a use method for biopsy needle visualization enhancement thereof.

### Related Art

In needle biopsy and interventional minimal invasive surgery of human organ tissues, ultrasonic high-frequency linear array probes or low frequency convex array probes are usually used for the guidance of biopsy needles and interventional needle heads. In China and United States, instead of using a puncture guide mounted on an ultrasonic probe, many clinicians operate according to their use experience to guide needle biopsy or guide an interventional needle head. When a needle tip is inside a human tissue, the clinicians would make a judgment by the subtle feeling of their fingers to the force passed from the needle tip when it is moving inside tissue, and the live image on the screen displayed by ultrasonic equipment.

During the operation, a doctor usually holds a transducer with one hand, places the transducer on the surface of the skin above a biopsy tissue or an interventional tissue, and then controls and manipulates the needle head with the other hand under real-time monitoring of the ultrasonic equipment. This operation is so difficult that usually only the most experienced clinician in an ultrasound department can perform it. The main reason for adopting this invention is that a performing doctor often cannot find the needle body and the needle head of a puncture or interventional needle in the ultrasonic image in actual operation, and can only operate according to the experience.

In terms of existing equipment, one of the main reasons for this problem is that: a regular high frequency linear array probe is usually operated at a high center frequency, such as 10 to 12 MHz, and has a generated effective acoustic field thinner in the elevation direction, the direction perpendicular to the azimuth direction (i.e. the probe element arrangement direction), thus forming a thin-wall shaped acoustic field that is longer in the probe element arrangement direction and thinner in the direction perpendicular to the element arrangement direction. Since most of the time, it is expected that the biopsy needle to be parallel to the probe element arrangement direction and fall into the thin-wall shaped effective acoustic field formed by the probe during ultrasonic imaging monitoring in the tissue needle biopsy and interventional surgery, the acoustic field thinner in elevation often tends to miss the puncture needle, and thus it is very hard for the doctor to capture the puncture needle with the effective acoustic field. This puts a very high skill requirement, both on the experience and technique of the performing doctor.

Through searching, the prior art discloses a puncture enhancement method (Application No.: 201510888869.9), including: in the current scanning with large steering angle ultrasound beams employed for enhanced display of the biopsy needle image, transmitting a plurality of specific waveform ultrasonic waves with different steering angles; identifying an insertion orientation of the puncture needle according to scanned image frame data corresponding to the plurality of specific waveform ultrasonic waves with different transmitting angles; adjusting a corresponding steering angle for a next round transmits of ultrasonic waves with a large steering angle according to the identified insertion orientation of the puncture needle, where the transmitting direction of the ultrasonic wave is perpendicular or approximately perpendicular to the identified insertion orientation of the puncture needle under the large steering angle.

A puncture needle enhancement system disclosed by this solution still uses a method for adjusting an angle of the ultrasonic probe to enhance the image acquisition effect of the needle. Actually, the doctor still needs to constantly search the needle during the operation, and the problem of the current ultrasonic probe has not been solved.

### SUMMARY

### 1. Technical Problem to be Solved by the Present Invention

The present invention aims to overcome the deficiency that a needle body and a needle tip of a biopsy or interventional needle often cannot be found in an ultrasonic image in the prior art, and provides an ultrasonic probe, as well as an ultrasonic imaging system and a use method thereof for biopsy needle visualization enhancement. Probe array elements added on the elevation direction of the ultrasonic probe in the present invention extends the thickness of an elevation direction vertical to a transducer element arrangement direction, thereby generating an elevation direction thickened effective wall shape ultrasonic acoustic field, and thus enhancing the visibility of the biopsy needle under ultrasonic real-time monitoring.

### 2. Technical Solution

In order to achieve the foregoing objective, the technical solution provided by the present invention is as follows:

an ultrasonic probe for biopsy needle visualization enhancement of the present invention includes:

a shell;

a center element array, used for generating an ultrasonic acoustic field and mounted inside the shell; and

lateral element arrays, mounted on the two sides of the center element array in parallel, where generated ultrasonic acoustic fields are superimposed with an ultrasonic acoustic field generated by the center element array to obtain a thicker ultrasonic acoustic field.

As a further improvement of the present invention, an element of the center element array is made from one of a piezoceramic material, a piezoceramic composite material, a capacitive micro electro mechanical ultrasonic transducer chip or a piezoceramic micro electro mechanical ultrasonic transducer chip, and an element of each lateral element array is made from one of a piezoceramic material, a piezoceramic composite material, a piezoceramic single-crystal material, a capacitive micro electro mechanical ultrasonic transducer chip or a piezoceramic micro electro mechanical ultrasonic transducer chip. In one case, the center element array and the lateral element arrays are capacitive micro electric mechanical ultrasonic transducers (CMUT). In another case, the center element array and the lateral element arrays are piezoceramic micro electromechanical ultrasonic transducers (PMUT).

As a further improvement of the present invention, the probe is a high frequency linear array probe or a convex array probe.

As a further improvement of the present invention, at least one lateral element array is arranged on each of two elevation sides of the center element array.

As a further improvement of the present invention, the number of elements of each lateral element array is equal to the number of elements of the center element array; and/or, an element pitch of each lateral element array is equal to an element pitch of the center element array.

As a further improvement of the present invention, the height of each element in each lateral element array is not greater than the height of each element in the center element array.

As a further improvement of the present invention, each lateral element array is provided with an independent control circuit capable of controlling a working state of the lateral element array manually or through an electronic signal.

As a further improvement of the present invention, a control switch is mounted on the shell and used for performing manual control of the working states of the lateral element arrays.

As a further improvement of the present invention, only the center element array is covered by an acoustic lens, or the center element array and the lateral element arrays are all covered by acoustic lenses.

As a further improvement of the present invention, the acoustic stack of each of the lateral element arrays is tilted with an outward steering angle relative to the center element array such that the lateral element array are opened outwards.

An ultrasonic imaging system of the present invention includes:

an ultrasonic transmitting module, used for generating a transmit pulse;

an ultrasonic probe, including a center element array and lateral element arrays, and used for transmitting the transmit pulse generated by the ultrasonic transmitting module in a form of an acoustic wave signal and receiving a returned acoustic wave signal, and converting the returned acoustic wave signal into a corresponding electronic signal;

an ultrasonic receiving module, used for receiving the electronic wave signal returned by the ultrasonic probe and performing signal processing and image display, where under certain conditions, the ultrasonic receiving module and the ultrasonic probe are directly connected integrated circuit chips, and the ultrasonic receiving module may also be directly used for receiving the returned acoustic wave signal; and

a user interface used for controlling a system control unit to perform a corresponding operation.

As a further improvement of the present invention, the ultrasonic transmitting module includes a transmit waveform generator which transmits a generated waveform to a transmit beam forming unit for corresponding focusing delay and then transmits to a pulse generator, and a transmit pulse is transmitted to the center element array and the lateral element arrays through a transmitting/receiving T/R unit.

As a further improvement of the present invention, the ultrasonic receiving module includes a receiving front end which amplifies the electronic signal converted from the acoustic wave signal and forms a digital signal through an A/D converter, dynamic focusing is performed on the converted digital signal in a received beam forming unit to form a received beam, and then the received beam passes through an mid-processing unit and an image post processing unit in sequence to form an ultrasonic image displayed on a display.

As a further improvement of the present invention, the lateral element arrays are provided with independent control circuits, an electronic signal generated by a lateral control unit controls working states of the lateral element arrays, and the lateral control unit is operated through the user interface or a control switch.

As a further improvement of the present invention, the ultrasonic transmitting module, the ultrasonic receiving module and the ultrasonic probe enable signal transmitting and signal receiving through the transmitting/receiving T/R unit, and the electronic signal generated by the lateral control unit controls the working states of the lateral element arrays by connecting or disconnecting the lateral element arrays with or from the transmitting/receiving T/R unit.

As a further improvement of the present invention, the system further includes an image analysis unit which acquires a real-time image from the post processing unit in the ultrasonic receiving module, identifies whether a needle body exists in the image, and if no needle body exists, sends a signal to the system control unit to turn the lateral element arrays into a working state, through the lateral control unit.

As a further improvement of the present invention, when the image analysis unit determines that the needle body exists in the image, the image analysis unit further determines whether the needle body is in an acoustic field of the center element array. If the answer is true, the system control unit sends a signal to the lateral control unit to turn off the lateral element arrays.

As a further improvement of the present invention, the image analysis unit determines whether the needle body appears per gray scale values and an object slenderness ratio in the ultrasonic image.

An use method of the ultrasonic imaging system of the present invention includes the following specific processes:

S01, turning on a center element array only to scan a target object under a normal mode to acquire a clear ultrasonic image;

S02, finding a target region for tissue needle biopsy or interventional surgery through real-time scanning;

S03, inserting a surgical needle into the target region of a human tissue;

S04, turning on lateral element arrays to get into a needle body capturing mode with an effective acoustic field thickened in the direction perpendicular to an array azimuth direction, so as to quickly find out and capture a needle body of the needle;

S05, manipulating a probe and the needle body to capture the needle body; and

S06, determining whether the needle body is found, and continuing the operation of SOS if the needle body is not found; and if the needle body is found, moving the ultrasonic probe such that the needle body moves toward the acoustic field generated by the center element array to complete the target capturing of the needle body.

As a further improvement of the present invention, the step S04 to the step S06 are completed through observation and manual control of the switch, or are automatically completed in the presence of an image analysis unit.

As a further improvement of the present invention, after the step S06, the use method further includes the steps of acquiring a clear image:

S07, turning off the lateral element arrays to make the lateral element arrays stop working, returning to a mode that only the center element array works, and observing the ultrasonic image;

S08, determining whether the needle body disappears in the image, and returning to the step S04 if the needle body disappears, or proceeding to the next step if the needle body exists; and

S09, when the needle body exists, continuing to scan for imaging, and simultaneously executing the step S08 for needle appearance determination.

As a further improvement of the present invention, the step S04 to the step S09 are completed through observation and manual control of the switch, or are automatically completed in the presence of an image analysis unit.

As a further improvement of the present invention, after the step S04, a process that the image analysis unit determines whether the needle body appears includes:

S1, binarizing the image through an image gray level threshold value predetermined through manual judgment or deep learning;

S2, performing target separation on the binarized ultrasonic image;

S3, analyzing separated targets, and searching a target with a slenderness ratio and a straightness value exceeding preset threshold values;

S4, sending the target satisfying the feature in S3 to a pattern recognition or artificial intelligence network for analysis to determine whether the target is a target needle body; and

S5, sending a corresponding signal indicating that the needle body is found or no needle body is found to a system control unit according to a result in S4.

In the present invention, in addition to the center element array of a regular probe, two or more ultrasonic element arrays are added onto the probe in elevation direction, a direction perpendicular to the probe element arrangement direction (i. e. the azimuth direction). Elements of these added lateral probe element arrays extend the elevation direction, thereby generating a laterally thickened effective wall shape ultrasonic acoustic field during imaging. The wall shape ultrasonic acoustic field is formed by transmitting ultrasonic beams on the elements from one end of the probe to the other end with a plurality of center points in the probe element arrangement direction. The cross section of the acoustic field in elevation direction, perpendicular to the element arrangement direction, has a hyperboloid shape. The added lateral probe arrays increase the thickness of the hyperboloid, thereby enlarging the effective range of the ultrasonic acoustic field, so that it is easier to capture a puncture needle that is parallel or approximately parallel to the ultrasonic probe element arrangement direction, i.e. the azimuth direction in actual operation. The lateral element arrays on both sides are separately controlled from the center element array of the ultrasonic probe, and may be turned on or off by a control button on a transducer handle. Therefore, an enhanced needle searching function with the arrays on both sides turned on may be selected to use, or may not be used. This selection may be switched during the process of the needle usage.

### 3. Beneficial Effects

Compared with the prior art, the adoption of the technical solutions provided by the present invention has the following beneficial effects:

the ultrasonic probe of the present invention has a plurality of lateral element arrays at side portions of the center element array, and the elements of the added lateral element arrays extend the width in elevation direction, the direction perpendicular to the transducer element arrangement direction, thereby generating the laterally thickened effective wall shape ultrasonic acoustic field during imaging, so that the needle body of the puncture needle may be captured more easily.

### BRIEF DESCRIPTION OF THE DRAWINGS

FIG. 1 is an illustration of monitoring a puncture needle by a high frequency linear array ultrasonic probe;

FIG. 2 is an illustration of a linear array probe having a plurality rows of element arrays capable of enhancing the visibility of a biopsy needle;

FIG. 3 is an example of visualization enhancement of a needle body in an ultrasonic effective acoustic field created by a plurality rows of element arrays;

FIG. 4 is an illustration of a vertical cross section of an acoustic field generated by elements of a center element array and lateral element arrays of an ultrasonic probe;

FIG. 5 is an illustration of a cross section of an acoustic field generated when the elements of the lateral element arrays have the same heights as center array elements;

FIG. 6 is an illustration of an arrangement of an acoustic lens on the ultrasonic probe;

FIG. 7 is an array arrangement method where included angles are formed between the lateral element arrays and the center array;

FIG. 8 is an illustration of a cross section of an acoustic field generated when included angles are formed between the lateral element arrays and the center element array;

FIG. 9 is an illustration of an ultrasonic probe provided with a lateral element array control switch;

FIG. 10 is a schematic diagram of an ultrasonic imaging system with independent control of lateral element arrays;

FIG. 11 is a schematic diagram of an ultrasonic imaging system with intelligent control of lateral element arrays;

FIG. 12 is a schematic diagram of a clinical operation workflow for searching a needle body of a biopsy or interventional needle;

FIG. 13 is a schematic diagram of an image analysis algorithm for searching a needle body of a biopsy or interventional needle; and

FIG. 14 is an illustration of a convex probe made with lateral element arrays.

Labels in the schematic diagrams: 100: transducer probe; 101: center element array; 102/103: lateral element array; 104: transmitting/receiving T/R unit; 105: pulse generator; 106: transmit beam forming unit; 107: waveform generator; 108: receiving front end; 109: A/D converter; 110: received beam forming unit; 111: mid-processing unit; 112: image post processing unit; 113: system control unit; 114: user interface; 115: display; 116: image analysis unit; 117: lateral control unit; 200: effective acoustic field domain; 201/202/203: effective acoustic field; 300: wall shape ultrasonic acoustic field; 400: needle body; 401/402/403: region of acoustic field; 500: needle plane; 600: control switch; 601/602: circuit switch; 700: acoustic lens; 800: ultrasonic image; 900: convex array probe; 901: center element array; 902/903: lateral element array; and 904: control button.

### DETAILED DESCRIPTION

For further understanding of the present invention, the present invention is described in detail with reference to the drawings and embodiments.

The structures, proportions, sizes, and the like depicted in the accompanying drawings of the specification merely serve to illustrate the disclosure of the specification to allow for reading and understanding by those skilled in the art, are not intended to limit the implementation of the present invention, and therefore do not constitute any substantial technical meaning. Any modification of a structure, alteration of a proportional relationship, or adjustment of a size shall still fall within the scope of the technical content disclosed in the present invention without affecting the effects and objectives of the present invention. In addition, terms such as “above”, “below”, “left”, “right”, “middle”, and the like in the specification are only used for the clarity of description, and are not intended to limit the implementation scope of the present invention. Without substantially changing the technical content, an alteration or adjustment of the relative relationship of such terms shall be construed as falling within the implementation scope of the present invention.

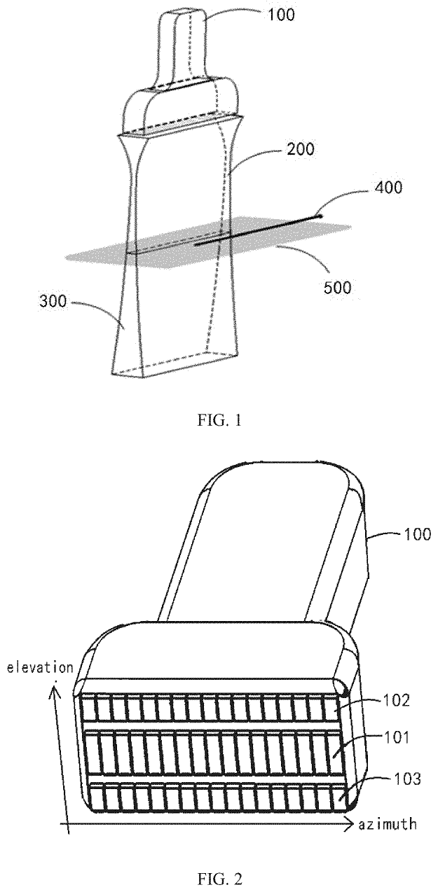

FIG. 1 illustrates an example that a high frequency linear array probe monitors a needle body of a biopsy needle in real time, but cannot find the needle body. During imaging, a transducer probe 100 of the high frequency linear array probe transmits a plurality of ultrasonic beams from left to right to a tissue below to form a wall shape ultrasonic acoustic field 300 which extends along the azimuth direction of the transducer probe 100, and has a hyperboloid cross section in the elevation direction. An effective acoustic field domain 200 of the wall shape ultrasonic acoustic field is defined by the out layer signal with signal strength at −30 dB below the maximum acoustic intensity. Objects within this effective acoustic field range may be clearly displayed in an ultrasonic image.

If the needle body 400 of the puncture needle falls completely or partially within this effective acoustic field domain 200, it will be displayed in a real-time image. In a tissue needle biopsy or interventional operation process, the biopsy needle 400 is generally parallel to the probe 100 azimuth/element arrangement direction. When the needle body 400 falls outside the range of the effective acoustic field domain 200, for example, when the needle body 400 is on a needle plane 500, but on the outer side of the effective acoustic field domain 200, it cannot be captured by the effective acoustic field, and thus is invisible in the formed ultrasonic image.

### Embodiment

With reference to FIG. 2, the basic structure of an ultrasonic probe for biopsy needle visualization enhancement of the present embodiment is the same as that of an existing probe, includes an external shell and elements mounted inside the shell. A plurality of elements is arranged in parallel to form a center element array 101. In addition, lateral element arrays are further mounted in the shell, and are placed on the two sides of the center element array 101. Ultrasonic acoustic fields generated by the lateral element arrays are superimposed with an ultrasonic acoustic field generated by the center element array 101 to obtain a thicker ultrasonic acoustic field.

The superimposition of the ultrasonic acoustic fields includes combination of the acoustic fields in a direction perpendicular to the element arrangement direction (the elevation direction), so that the spatial thickness is increased, thus, a better visualization of the puncture needle body 400 parallel to the azimuth direction of the ultrasonic acoustic field.

In one implementation, only one lateral element array may be added on one side of the center element array 101, which has a certain effect of thickening the ultrasonic acoustic field.

As a preference, at least one lateral element array may be mounted on each side of the center element array 101 to enhance the visualization of the puncture needle.

FIG. 2 illustrates an implementation of a multi-row linear array probe that enhances the visualization of the puncture needle, including a lateral element array 102 mounted on an upper side of the center element array 101 and a lateral element array 103 mounted on a lower side of the center element array. The lateral element arrays located on the two sides of the center element array 101 and the center element array 101 may have the same number of elements, and may have different or same element pitch, preferably same element pitch.

In a coordinate system of FIG. 2, an azimuth direction is an element arrangement direction in an array, and an elevation direction is perpendicular to the element arrangement direction, and also refers to a direction perpendicular to the side wall of the probe. A plurality of element arrays is distributed along the elevation direction.

The height h of each element in the lateral element array 102 and the lateral element array 103 may be equal to or less than the height of each element in the center element array 101. The height refers to a length in the elevation direction, perpendicular to the side wall of the transducer probe 100 or the element arrangement direction. In addition, the lateral element arrays and the center element array of the ultrasonic transducer probe may be made of the same material, such as one of a piezoceramic material or a piezoceramic composite material or a piezoceramic single-crystal material. The lateral element arrays and the center element array 101 may be made of different materials. For example, the center element array 101 is made of the piezoceramic single-crystal material, while the lateral element arrays on two sides are made of the piezoceramic material or the piezoceramic composite material or the like. In another embodiment, the center element array and the lateral element arrays are capacitive micro electromechanical ultrasonic transducers (CMUT) or piezoceramic micro electromechanical ultrasonic transducers (PMUT).

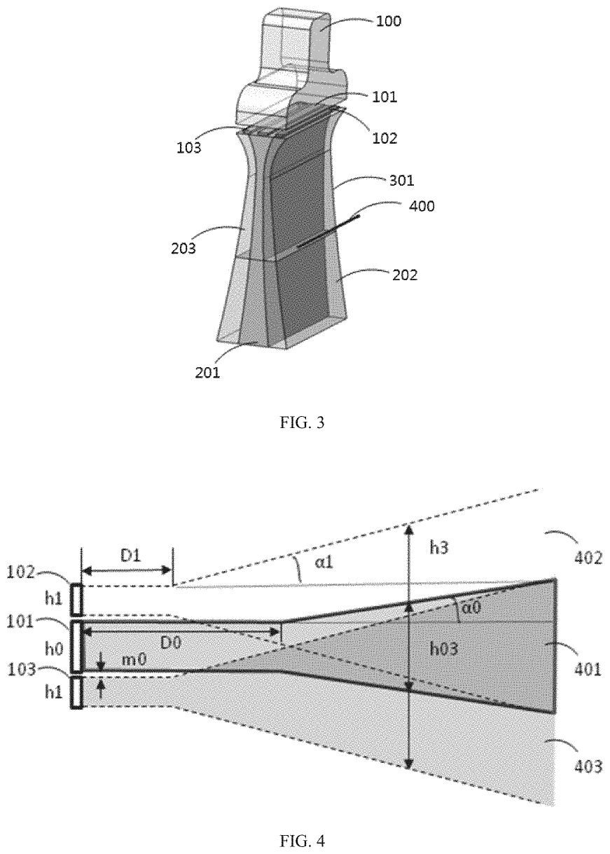

FIG. 3 illustrates an example of visual enhancement of a needle body in an ultrasonic effective acoustic field under a plurality of rows of element arrays. The effective acoustic field includes extra acoustic fields generated by the two rows of lateral element array 102 and lateral element array 103 of an ultrasonic transducer. When the ultrasonic probe is in an imaging state, if the lateral element arrays are both turned on to be in the imaging state, the lateral element array 102 will generate an extra effective ultrasonic acoustic field 202, and the lateral element array 103 will generate an extra effective ultrasonic acoustic field 203, in addition to an effective ultrasonic acoustic field 201 generated by the center element array 101, thus forming an ultrasonic acoustic field 300 with superimposition effect.

As shown in FIG. 3, these extra ultrasonic effective acoustic fields 202 and 203 are combined with the effective acoustic field 201 generated by the center element array 101 to form a combined effective acoustic field that is thicker than the effective acoustic field 201 generated by the center element array 101 alone in the elevation direction, i.e., a lateral direction perpendicular to the element arrangement direction. Specifically, an acoustic field thickness increased by the lateral acoustic fields in the elevation direction may be calculated according to the element height in each element array.

FIG. 4 illustrates cross sections in the elevation direction of 3 dB acoustic fields generated by the elements of the three rows of arrays of ultrasonic transducer probes in FIG. 2 without extra focusing of an acoustic lens. In FIG. 4, the element height of the center element array 101 is h0, and the element height of the lateral element arrays 102 and 103 is h1. A spacing between the center element array 101 and each lateral element array is m0. Cross sections in elevation direction of the 3 dB acoustic fields generated by the elements of the three element arrays are shown as acoustic field regions 401, 402 and 403 respectively.

The elements of the center element array are center elements, and a near field region of the 3 dB acoustic field generated by the center elements has a depth: D0=h0\hat{ }2/(4*wavelength). In the near field region, the width of the 3 dB acoustic field generated by the center elements is equal to the height h0. Later, the acoustic field then diverges with a divergent flare angle: α0=arcsin(0.61*2*wavelength/h0), where wavelength refers to a wavelength of acoustic waves. Correspondingly, near field regions of the 3 dB acoustic fields generated by the lateral element arrays have depths: D1=D2=h1\hat{ }2/(4*wavelength). Later, the acoustic fields then diverse with a divergent flare angle: α1=arcsin(0.61*2*wavelength/h1).

Assuming that a center frequency of a transmit waveform of the probe is 8 MHz, the wavelength is equal to 0.2 mm, the element height h0 of the center element array of the probe is equal to 4 mm, the element height h1 of the two lateral element arrays is equal to 3 mm, and then the 3 dB near field of the elements of the center element array 101 has the depth of 2 cm and the divergent flare angle α0 of 3.5 degrees. The 3 dB near fields of the elements of the two lateral element arrays have the depth of 1.13 cm and the divergent flare angle α1 of 4.7 degrees.

It can be seen that the adding of the elements of the two rows of lateral element arrays rapidly extends the 3 dB combined effective acoustic field in the elevation direction: within the depth D1, h0 is increased to h0+2*h1+2*m0. Usually, m0 is relatively small and may be ignored. Beyond the depth D1, the thickness of the 3 dB acoustic field with any depth D is H=(D−D1)×tan(α1)+2*h1+h0. The probe in the example is taken as an example. In FIG. 4, at the depth of 3 cm, the 3 dB acoustic field has a thickness h3 of 1.15 cm in the elevation direction. While if there is only the center element array, the thickness h03 of the 3 dB acoustic field with this depth in the elevation direction is only 4.6 mm, which is only one third of the thickness of the superimposed acoustic field.

As mentioned above, in the tissue needle biopsy or interventional surgery, the needle body 400 of the puncture or surgical needle is usually parallel to the element arrangement direction of the ultrasonic transducer to obtain a better observation angle. In this case, a thicker volume in elevation direction will help the needle to be captured more easily by the ultrasonic effective acoustic field in a needle biopsy guiding process. If handled and operated properly, during the procedure of real time ultrasound monitoring of tissue needle biopsy and interventional surgery, this will greatly increase the sensitivity to detect and visualize the biopsy/interventional surgery needle 400.

In FIG. 3, the needle body 400 shows up in the newly add on acoustic field region 202 generated by the extra row array in elevation direction, demonstrated as the shadowed region, but not in the original central ultrasound acoustic field 201 generated by central row 101. The new acoustic field generated by the newly added lateral element array 102 increases the probability that the puncture needle is captured and displayed in the ultrasonic image.

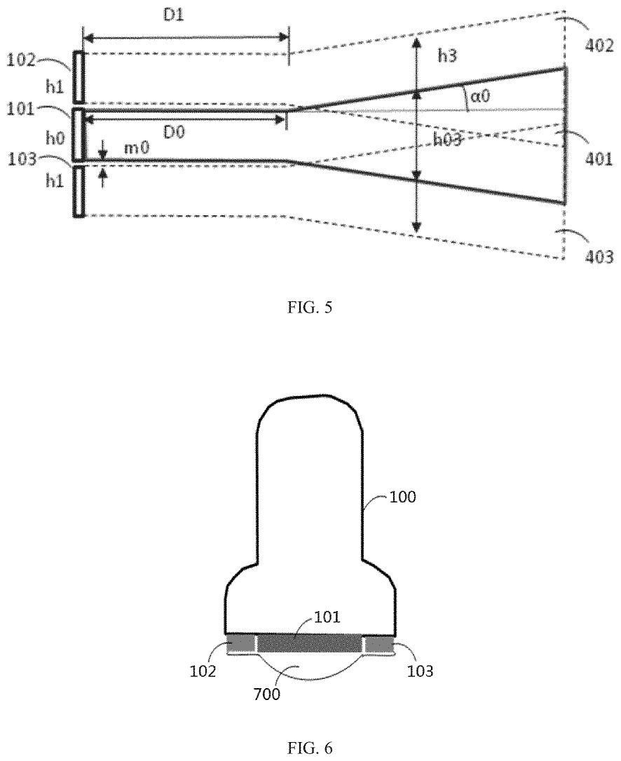

FIG. 5 illustrates a cross section of an acoustic field generated when the elements of the lateral element arrays have the same height as the center elements. The elements of the center element array are used as the center elements, and the elements of the lateral element arrays are used as lateral elements. When the height of each lateral element is equal to the height of the center element, i.e., h0=h1, the length D1 of the near field region is equal to D0, and the thickness of the lateral element in the elevation direction of the 3 dB acoustic field is also h03. Under this condition, the increased acoustic field thickness h3 resulted from the combination of the 3 dB acoustic field of the lateral elements with the acoustic field of the center element array is less than the increased acoustic field thickness h3 resulted from the condition where the height of the lateral element arrays is less than the height of the center element array (i.e., h1<h0, the increased thickness is h3 shown in FIG. 4). Finally, the thickness h03+h3+h3 generated by superimposing all the acoustic fields is reduced to some extent relative to the adoption of lateral element arrays with a smaller height, but the thickness of the lateral acoustic field is still increased.

In the similar way, when the element height of the used lateral element arrays is greater than the element height of the center element array, the thickness of the acoustic field may also be increased within a certain range, and the probability that the puncture needle is captured and displayed in the ultrasonic image may be increased.

In the present embodiment, the number of the elements in each lateral element array may be less than the number of the elements in the center element array. As another embodiment, the element pitch of the lateral element arrays may be greater than the element pitch of the center element array. When the length of each lateral element array is equal to the length of the center element array, a relatively smaller number of elements will inevitably increase the element pitch. If the length of each lateral element array is not equal to the length of the center element array, when the lateral element array includes a relatively smaller number of elements, it is possible that the element pitch is also smaller. The present embodiment is mainly intended to find the needle body of the needle more quickly by using the acoustic fields generated by the lateral element arrays. After the needle body is captured, the probe may further be switched to use the center element array only for clearer images, so that there is no particular limitation to the number of the elements and the element pitch of each lateral element array as long as the acoustic field of the lateral element array may generate an acoustic wave signal to find the needle quickly.

As shown in FIG. 6, in specific implementation, the lateral element arrays on two sides may not use acoustic lenses, thus generating a thicker acoustic field in the elevation direction. In FIG. 6, the center element array 101 is provided with an acoustic lens 700, and the two rows of lateral element arrays are not provided with lenses.

Of course, in another implementation manner, the center element array 101 and the lateral element arrays 102 and 103 on the two sides may be all covered by the acoustic lens.

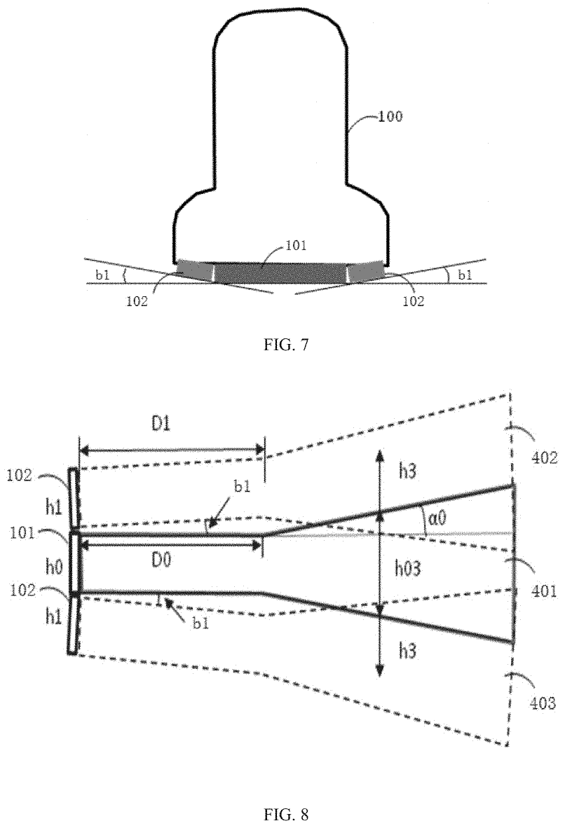

In a further implementation manner, in order to further increase the thicknesses of the lateral acoustic fields, an outwards tilting included angle is formed between the surface of each of the lateral element arrays on the two sides and the surface of the center element array. As shown in FIG. 7, an included angle b1 is formed between the surface of each lateral element array 102/103 and the center element array 101, so that acoustic fields generated by the lateral element arrays 102 and 103 on the two sides are deflected toward a direction away from the acoustic field of the center element array 101. As shown in FIG. 8, the principal axes of the acoustic fields 402 and 403 generated by the two lateral element arrays 102 and 103 are flared outside to form the included angles b1 in comparison to the acoustic field generated by the center element array 101, thus increasing the thicknesses of the lateral acoustic fields. In manufacturing, this can be done in a special process to mount the side acoustic stacks 102 with predetermined out-warding angles.

Generally, a wider acoustic field in elevation direction which is perpendicular to the element arrangement direction of an ultrasonic probe array may often lead to a relatively low spatial resolution of an image, and the image will be blurry. This is due to the fundamental that the image pixel at a certain depth and lateral spatial location is formed by the summation of the tissue signals of the resolution cell volume centered at that spatial location. A thick elevation volume often results in lower image spatial resolution and a more haze like image, thus worse contrast resolution as more tissue are integrated inside this volume and contributes to the final reflected signal.

In order to avoid degradation of the contrast resolution of the image, the present embodiment will add one separate control for the lateral element arrays, that is, the lateral element arrays are provided with independent control circuits. The two rows of lateral element arrays in addition to the center element array are turned on only when necessary, to form the thicker effective acoustic field in the elevation direction.

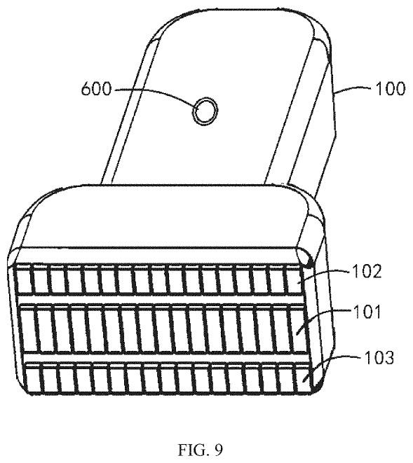

FIG. 9 illustrates an embodiment for manual control of the lateral element arrays working states. A control switch 600 is mounted on the shell of the transducer probe 100, and the control switch 600 may be a button or a knob. The button is taken as an example. When a user needs to turn on the lateral element arrays on the two sides, the user may press this button, and the system will turn on the two rows of arrays to form the thickened wall shape ultrasonic effective acoustic field. When it is not necessary, this button only needs to be pressed again, and the system will turn off the lateral arrays.

In another implementation manner, the turning on and turning off of the lateral element arrays 102 and 103 of the probe are controlled by an electronic signal. A system control unit sends the signal to control whether the lateral element arrays are on or off.

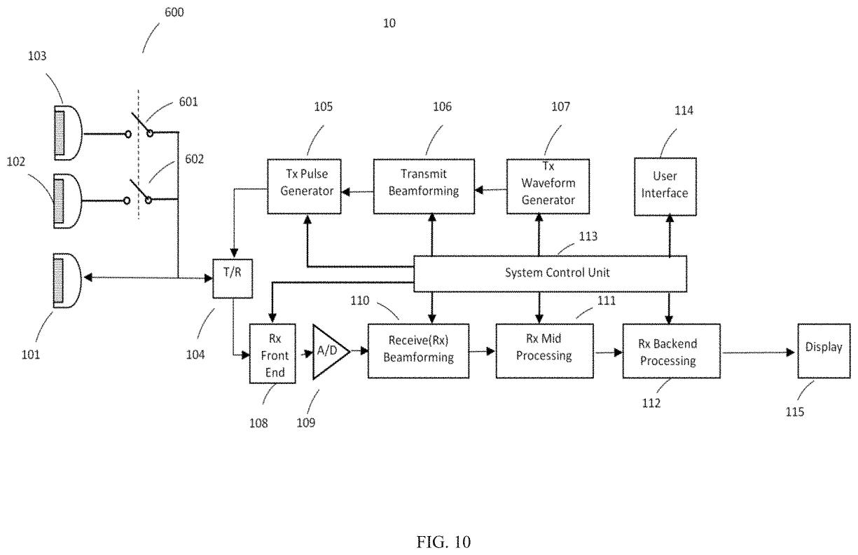

FIG. 10 illustrates an ultrasonic imaging system using an ultrasonic probe with independent control of the lateral element arrays. The ultrasonic imaging system includes an ultrasonic transmitting module, used for generating a transmit pulse; an ultrasonic probe, including a center element array and lateral element arrays, and used for transmitting the transmit pulse generated by the ultrasonic transmitting module in a form of an acoustic wave signal, and receiving a reflected acoustic wave signal and converting the reflected acoustic wave signal into a corresponding electronic signal; an ultrasonic receiving module, used for receiving the electronic wave signal received by the ultrasonic probe and performing signal processing and image display, and an user interface, used for controlling a system control unit to perform a corresponding operation.

As shown in FIG. 10, the ultrasonic transmitting module includes a waveform generator 107. This unit generates a transmit waveform, and transmits the generated waveform to a transmit beam forming unit 106 for transmitting time delays and then to a pulse generator 105. The detail operations and waveform transmissions of the pulse generator 105, the transmit beam forming unit 106 and the waveform generator 107 are all controlled by the system control unit 113. Generated transmit pulses of various channels are sent to a transmit/receive T/R unit, i.e., a transmit-receive switch unit. The T/R unit 104 sends the transmit pulses of the various channels to the various element arrays including the center element array 101 and the two lateral element arrays.

A circuit switch 602 is installed on an element circuit leading to the lateral element array 102. A circuit switch 601 is installed on an element circuit leading to the lateral element array 103. The circuit switches 601 and 602 are simultaneously controlled by the control switch. When the button of the control switch 600 is pressed by an operation doctor, the lateral element arrays 102 and 103 are turned on. At this time, the transmit pulse sent from the T/R unit 104 will be simultaneously sent to corresponding elements in the center element array and the lateral element arrays, and after tissue reflected echo signals received by the elements in the center element array and the lateral element arrays are converted into corresponding electronic signals, the electronic signals are converged in the T/R unit 104. A naturally synthesized signal will be sent to an analog signal receiving front end 108 through the T/R unit 104. In this case, system 20 is working on the needle searching mode, generate a much thicker acoustic field in elevation direction to facilitate the needle finding.

If the control switch 600 is not pressed, the transmit pulse is only sent to the center element array 101, and correspondingly, only the electronic signal converted from the tissue echo signal received by the center element array 101 is sent to the T/R unit 104 and then to the analog signal receiving front end 108 for signal amplification. The echo signal is amplified and filtered at the analog signal receiving front end 108, and then sent to an A/D converter 109 to be converted into a digital signal. In this case, the system works in a normal ultrasonic imaging mode, and the image detail and contrast resolution are relatively high.

Per the development of the chip technology, the analog signal receiving front end 108 and the A/D converter 109 are usually integrated in one chip unit. The converted digital signals are dynamically focused at a receive beam forming unit 110 to form a received beam. The received beam finally forms a display image displayed on a display 115 through a subsequent intermediate processing unit 111 and an image post processing unit 112.

It should be noted that units starting from the received beam forming unit 110 and the system control unit 113 may be implemented on a large-scale field programmable logic gate array (FPGA) and a digital signal processing chip DSP, and also may be implemented on a personal computer (PC) or implemented in an embedded system. It may also be the case that one part is implemented on the FPGA and the DSP, and the other part is implemented on the PC or the embedded system. In this system, the lateral element arrays are turned on and turned off through the control switch 600. Usually, if the button corresponding to the control switch 600 is operated once, the lateral element arrays will be connected to the T/R unit. When the button is pressed again, the T/R unit will be disconnected from the lateral element arrays.

In addition, a lateral control unit 117 may further be used to send an electronic signal to control the circuit switches 601 and 602, and the corresponding control switch 600 is used to enable the lateral control unit 117 to generate the corresponding electronic signal.

In another system implementation manner, the lateral element arrays 102 and 103 of the probe are automatically turned on and turned off per the result of image analysis by the system control unit.

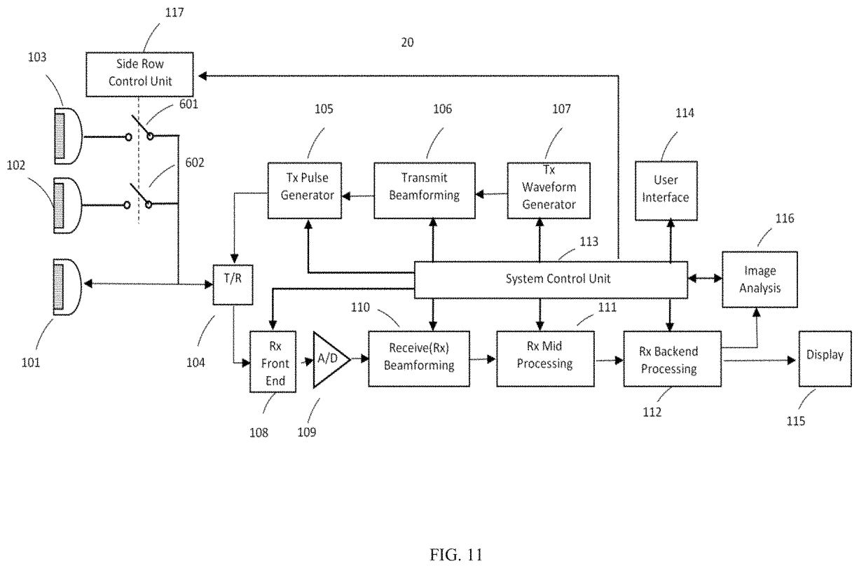

FIG. 11 illustrates an implementation example of the ultrasonic system. In this system, a user manipulates the system control unit 113 through the user interface 114, so that the system enters into a needle head guidance working mode for tissue biopsy or interventional surgery. In this mode, the system control unit may turn on an image analysis unit 116, and send a real-time ultrasonic image from the image postprocessing unit 112 to the image analysis unit 116. This unit may identify whether a puncture needle appears in the image based on artificial intelligent image analysis or image pattern recognition.

If the needle body 400 of the puncture needle does not appear in the real-time ultrasonic image, the image analysis unit 116 feeds the information to the system control unit 113, and the system control unit 113 sends an instruction to the lateral control unit 117 to inform the lateral control unit to turn on the lateral arrays, and to generate the thickened wall shape effective ultrasonic acoustic field, so that the system is in a needle body searching mode to better find the puncture needle body. When the needle body of the puncture needle is captured by the ultrasonic acoustic field of the probe, and a relatively strong echo is formed in the ultrasonic image, the image analysis unit 116 may determine whether the needle body has been in an effective acoustic field formed by elements of the center element array 101 according to a preset threshold. Usually, if the needle body is within the acoustic field range of the center element array, a generated echo signal is relatively strong. The specific intensity may be determined according to empirical values. If a determination result is true, the system considers that the needle body 400 of the puncture needle may still be captured even if the lateral arrays are turned off, and the image analysis unit 116 sends a result to the system control unit 113, and the system control unit 113 sends a signal to the lateral control unit 117 to enable the lateral control unit to turn off the lateral arrays 102 and 103, thus making the image in a high-resolution normal imaging mode.

In the ultrasonic imaging system of the ultrasonic probe with the lateral element arrays in FIG. 11, the puncture needle body is specifically identified based on if there is a strong echo object shown up in the image with high slenderness ratio. The echo intensity, denoted by a gray scale, of the object in the ultrasonic image and a slenderness ratio of the object per se are both used to determine whether the puncture needle body appears in the image.

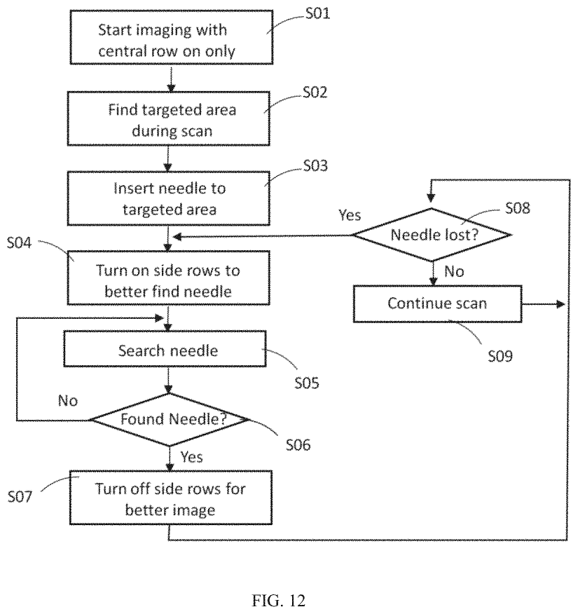

FIG. 12 illustrates a schematic diagram of a real-time operation workflow using an ultrasonic probe with a plurality of lateral arrays, and the associated imaging system in actual clinical environment. For the foregoing ultrasonic imaging system, a specific use method thereof is that:

S0, a center element array 101 is turned on only to scan a target object under a normal imaging mode to acquire a clear ultrasonic image;

in real-time application, a clinician may firstly turn on elements of the center element array 101 only to scan the target object under a normal high-resolution mode to acquire the ultrasonic image with higher contrast resolution;

S02, a target region for tissue needle biopsy or interventional surgery is found out through real-time scanning;

S03, a surgical needle is inserted into the target region of a human tissue;

S04, lateral element arrays are turned on to get into a needle body capturing mode with an enlarged effective acoustic field, so as to quickly find out and capture a needle body of the needle;

as mentioned above, in this mode, in an elevation direction, the field of view of an acoustic field of the ultrasonic probe is greatly expanded, so that the needle body of the puncture or interventional needle inserted in a direction roughly parallel to the main direction of the wall shape acoustic field, namely the element arrangement direction, or the azimuth direction, may be better observed, and the needle body of the needle is captured more easily.

S05, the probe and the needle body are manipulated to capture the needle body; and

S06, to determine if the needle body is found or not, and the operation of S05 is continued if the needle body is not found; if the needle body is found, the ultrasonic probe is moved toward the region such that acoustic field generated by the center element array can completely capture of the needle body target.

In the steps S05 and S06, the doctor searches the needle in this mode. The doctor manipulates the probe and the needle body, so that after the needle body is captured and displayed in the image, the doctor may move the ultrasonic probe to enable the needle body to enter into the acoustic field generated by the center element array of the probe, and the needle body is more clearly shown.

In the steps S04 to S06, the doctor turns on and turns off the lateral element arrays by manual control of switch 600, and observes whether the needle body is captured through a display. The doctor may further control the lateral element arrays to be turned on and turned off through a user interface, and observe whether the needle body is captured through the display.

In addition, if it is desired to acquire a better real-time image for puncture or interventional surgery monitoring, the method may further include the following operations after the step S06:

S07, the control switch is pressed again to turn off the lateral element arrays to make the lateral element arrays stop working, a mode that only the center element array works is recovered, and the ultrasonic image is observed;

S08, whether the needle body disappears in the image is determined, and the operation returns to the step S04 if the needle body disappears, or the next step is executed if the needle body exists; and

S09, when the needle body exists, continue the imaging scan, at the same time, the step S08 is executed to continuously determine if the needle body appears in the image.

After the lateral arrays are turned off, if the needle cannot be found in real-time scanning as defined in step S08, the doctor may return to the step S04 to turn on the lateral arrays again, so as to capture the needle body again and achieve a better display of the needle body. If the needle body is in the field of view range, the doctor may continue to move the needle body, and monitor the puncture or interventional surgery by only turning on the center element array.

If the needle body is obvious in the step S08, the doctor may continue to guide the needle body in real time in the tissue biopsy or interventional surgery only with the center element array to complete the surgery in the step S09.

It is worth mentioning that although the control on the central element array and the lateral element arrays of the ultrasonic probe from the step S04 to the step S09 in this example is done manually by the doctor, according to the example shown in FIG. 9, the step S04 to the step S09 may be all automatically executed by the system with the participation of the image analysis unit, so that the doctor may concentrate on the real-time guidance of the puncture or interventional needle body.

To identify whether the needle body of the puncture needle appears in the image, the present invention provides an image analysis method such as Pattern Recognition for searching the needle body.

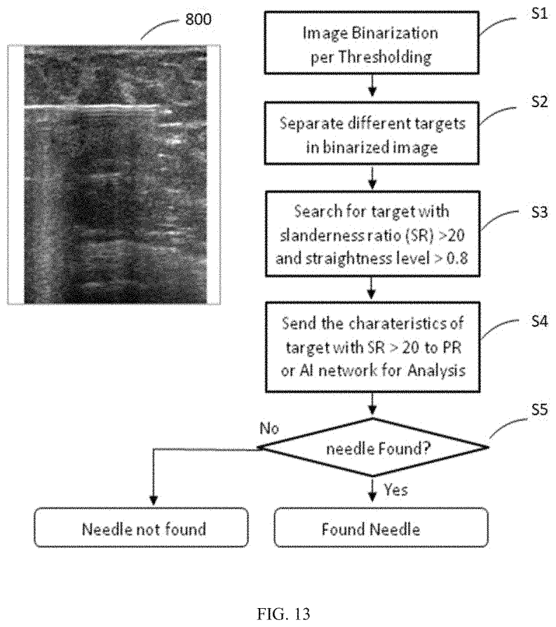

FIG. 13 illustrates an implementation flow chart of an image analysis algorithm in the image analysis unit. The ultrasonic image 800 is a real ultrasonic image for puncture monitoring, in which a white long strip is the captured puncture needle body.

In FIG. 13, the process that the image analysis unit determines whether the needle body appears is:

S1, the image is binarized through an image gray level threshold predetermined through empirical judgment or deep learning;

S2, target separation is performed on the binarized ultrasonic image;

the target separation may include a plurality of image processing steps, such as image filtering, feature extraction and image segmentation, so as to cluster and integrate objects in the image, resulted in a plurality of separated targets;

S3, the separated targets are analyzed, and a target with a slenderness ratio and straightness value exceeding set values is searched;

the slenderness ratio is equal to length divided by average width, and the straightness is equal to I-maximum width change/length;

S4, the target satisfying the feature in S3 is sent to a pattern recognition or artificial intelligence network for analysis to determine whether the target is a target needle body; and

S5, a corresponding signal indicating that the needle body is found or no needle body is found is sent to a system control unit according to a result in S4.

If the needle body is found, the image analysis unit 116 may send a signal indicating that the needle body is found to the system control unit 113, or may inform the system control unit 113 that the needle body is not found.

In the step S1, the image is binarized based on an image gray level threshold predetermined according to the experience or deep learning, and the target satisfying this feature is sent to the pattern recognition or artificial intelligence network for analysis in the step S4, so as to determine whether the target is the needle body for puncture or intervention. The result is sent to a determiner S5. If the needle body is found, the image analysis unit 116 may send the signal indicating that the needle body is found to the system control unit 113, and otherwise may inform the system control unit 113 that the needle body is not found.

For the pattern recognition and the artificial intelligence network, feature analysis may be employed to make a judgment of the eligible target. This technique may be implemented through an existing program, and descriptions thereof are omitted.

In the present invention, the above introduction takes a high frequency linear array probe as an example. In engineering practice, the present invention may further be conveniently used for a convex array probe, so as to better find the puncture or interventional needle body in liver/kidney tissue needle biopsy or abdominal interventional surgery.

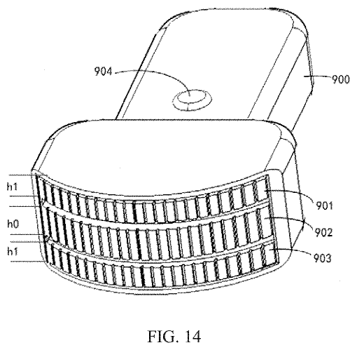

FIG. 14 illustrates a convex array probe 900 using a plurality of lateral element arrays of the present invention. The convex array probe 900 has three rows of element arrays, including a center element array 901, a lateral element array 902 and a lateral element array 903 in elevation direction, a direction perpendicular to element arrangement direction, and a control button 904. The number of elements of the lateral element arrays 902 and 903 is equal to the number of elements of the center element array 901. The element height h1 of the lateral element arrays may be equal to or less than the element height h0 of the center element array 901, or in some implementation manners, h1 is greater than h0. The imaging and imaging control methods of the convex array probe 900 are basically consistent with the aforementioned imaging control method of the high frequency linear array probe with a plurality of lateral arrays.

It should be noted that although the present invention only mentions that two rows of element arrays are added laterally, actually, a plurality of rows of element arrays may be added as needed, such as five rows and seven rows. In order to further improve the visual effect of the ultrasonic probe on the puncture and interventional surgery needles, in another implementation, the elements of the lateral arrays may also have different center frequencies, so that the lateral element arrays may have different element pitch and even different numbers of elements. Therefore, the effective thickness of the wall shape ultrasonic acoustic field generated by the probe is increased as much as possible to enable the acoustic field to capture the puncture needle body parallel to the main direction of the acoustic field more easily.

Although the present invention and implementations thereof have been exemplarily described above, the description is not limiting, the content shown in the accompanying drawings is merely one of the implementations of the present invention, and the actual structure is not limited thereto. Therefore, under the teaching of the present invention, any structures and embodiments similar to the technical solution that are made by those skilled in the art without creative efforts and without departing from the spirit of the present invention shall all fall within the protection scope of the present invention.

1. An ultrasonic probe for biopsy needle visualization enhancement, comprising:
a shell;
a center element array, used for generating an ultrasonic acoustic field and mounted inside the shell; and
lateral element arrays, mounted on the two sides of the center element array in parallel, generating ultrasound acoustic field, wherein generated ultrasonic acoustic fields are superimposed with the ultrasonic acoustic field generated by the center element array to obtain a laterally thicker ultrasonic acoustic field.

2. The ultrasonic probe for biopsy needle visualization enhancement according to claim 1, wherein an element of the center element array is made from one of a piezoceramic material, a piezoceramic composite material, a capacitive micro electro mechanical ultrasonic transducer chip or a piezoceramic micro electro mechanical ultrasonic transducer chip;
and an element of each lateral element array is made from one of a piezoceramic material, a piezoceramic composite material, a piezoceramic single-crystal material, or a capacitive micro electro mechanical ultrasonic transducer chip or a piezoceramic micro electro mechanical ultrasonic transducer chip.

3. The ultrasonic probe for biopsy needle visualization enhancement according to claim 1, wherein the probe is a high frequency linear array probe or a convex array probe.

4. The ultrasonic probe for biopsy needle visualization enhancement according to claim 1, wherein at least one lateral element array is arranged on each of two elevation sides of the center element array.

5. The ultrasonic probe for biopsy needle visualization enhancement according to claim 4, wherein the number of elements of each lateral element array is equal to the number of elements of the center element array; and/or, the element pitch of each lateral element array is equal to the element pitch of the center element array.

6. The ultrasonic probe for biopsy needle visualization enhancement according to claim 4, wherein the height of each element in each lateral element array is not greater than the height of each element in the center element array.

7. The ultrasonic probe for biopsy needle visualization enhancement according to claim 4, wherein each lateral element array is provided with an independent control circuit capable of controlling a working state of the lateral element array manually or through an electronic signal.

8. The ultrasonic probe for biopsy needle visualization enhancement according to claim 7, wherein a control switch is mounted on the shell and used for performing manual control of the working states of the lateral element arrays.

9. The ultrasonic probe for biopsy needle visualization enhancement according to claim 1, wherein only the center element array is covered by an acoustic lens, or the center element array and the lateral element arrays are all covered by acoustic lenses.

10. The ultrasonic probe for biopsy needle visualization enhancement according to claim 1, wherein each of the lateral element arrays is mounted in a tilted angle relative to the center element array to form an outward steering angle.

11. An ultrasonic imaging system, comprising:
an ultrasonic transmitting module, used for generating a transmit pulse;
an ultrasonic probe, comprising a center element array and lateral element arrays, and used for transmitting the transmit pulse generated by the ultrasonic transmitting module in a form of an acoustic wave signal and receiving a returned acoustic wave signal, and converting the returned acoustic wave signal into a corresponding electronic signal;
an ultrasonic receiving module, used for receiving the electronic signal returned by the ultrasonic probe and performing signal processing and image display; and
a user interface, used for controlling a system control unit to perform a corresponding operation.

12. The ultrasonic imaging system according to claim 11, wherein the ultrasonic transmitting module comprises a transmit waveform generator which sends a generated waveform to a transmit beam forming unit for corresponding focusing delay and then sends to a pulse generator, and then a transmit pulse is transmitted to the center element array and the lateral element arrays through a transmitting/receiving T/R unit.

13. The ultrasonic imaging system according to claim 11, wherein the ultrasonic receiving module comprises a receiving front end which amplifies the electronic signal converted from the acoustic wave signal and forms a digital signal through an A/D converter, dynamic focusing is performed on the converted multi-channel digital signal in a received beam forming unit to form a received beam, and then the received beam passes through a mid-processing unit and an image post processing unit in sequence to form an ultrasonic image displayed on a display.

14. The ultrasonic imaging system according to claim 11, wherein the lateral element arrays are provided with independent control circuits, an electronic signal generated by a lateral control unit controls the working states of the lateral element arrays, and the lateral control unit is operated through the user interface or a control switch.

15. The ultrasonic imaging system according to claim 14, wherein the ultrasonic transmitting module, the ultrasonic receiving module and the ultrasonic probe transmit and receive signal through the transmitting/receiving T/R unit; and the electronic signal generated by the lateral control unit controls the working states of the lateral element arrays by connecting or disconnecting the lateral element arrays with or from the transmitting/receiving T/R unit.

16. The ultrasonic imaging system according to claim 11, wherein the system further comprises an image analysis unit which acquires a real-time image from the post processing unit in the ultrasonic receiving module, identifies whether a needle body exists in the image, and if no needle body exists, sends a signal to the system control unit to turn the lateral element arrays into a working state, through the lateral control unit.

17. The ultrasonic imaging system according to claim 16, wherein when the image analysis unit determines that the needle body exists in the image, the image analysis unit further determines whether the needle body is in an acoustic field of the center element array; and if the answer is true, the system control unit sends a signal to the lateral control unit to turn the lateral element arrays off into a nonworking state.

18. The ultrasonic imaging system according to claim 17, wherein the image analysis unit determines whether the needle body appears through an object gray scale values and an object slenderness ratio in the ultrasonic image.

19. An operating method of an ultrasonic imaging system, comprising the following specific processes:
S01, turning on a center element array only to scan a target object under a normal imaging mode to acquire a clear ultrasonic image;
S02, finding a target region for tissue needle biopsy or interventional surgery through real-time scanning;
S03, inserting a surgical needle into the target region of a human tissue;
S04, turning on lateral element arrays to get into a needle body capturing mode with a thickened effective acoustic field wherein the thickened direction is perpendicular to the array element arrangement direction, so as to quickly find and capture a needle body of the needle;
S05, manipulating the probe and the needle body to capture the needle body; and
S06, determining whether the needle body is found, and continuing the operation of S05 if the needle body is not found; and if the needle body is found, moving the ultrasonic probe such that the needle body moves toward an acoustic field generated by the center element array, to complete the capturing of the target needle body.

20. The use method of the ultrasonic imaging system according to claim 19, wherein the step S04 to step S06 are executed through observation and the manual control of the switch, or are automatically executed in the presence of an image analysis unit.

21. The use method of the ultrasonic imaging system according to claim 19, wherein after the step S06, the operating method further comprises the steps of acquiring a clear image:
S07, turning off the lateral element arrays to make the lateral element arrays stop working, recovering a state that only the center element array works, and observing the ultrasonic image;
S08, determining whether the needle body disappears in the image, and returning to the step S04 if the needle body disappears, or proceeding to the next step if the needle body exists; and
S09, when the needle body exists, continuing to scan for imaging, and simultaneously executing the step S08 for needle appearance determination.

22. The operating method of the ultrasonic imaging system according to claim 21, wherein the step S04 to the step S09 are executed through observation and manual control of the switch, or are automatically executed in the presence of an image analysis unit.

23. The operating method of the ultrasonic imaging system according to claim 19, wherein after the step S04, a process that the image analysis unit determines whether the needle body appears comprises:
S1, binarizing the image through an image gray level threshold predetermined through manual judgment or deep learning;
S2, performing target separation on the binarized ultrasonic image;
S3, analyzing separated targets, and searching a target with a slenderness ratio and straightness exceeding set threshold values;
S4, sending the target that satisfies the feature defined in S3 to a pattern recognition or artificial intelligence network for analysis to determine whether the target is a target needle body; and
S5, sending a corresponding signal indicating that the needle body is found or no needle body is found to a system control unit according to a result in S4.
